(19) United States
(12) Patent Application Publication
(10) Pub. No.: US 2017/0174538 A1
(43) Pub. Date: Jun. 22, 2017

(54) Hydrogen Generation Device

(71) Applicants: Michael Lelah, Arlington Heights, IL (US); Paul Mulhauser, New York, NY (US); Steven A. Rye, Cape Coral, FL (US)

(72) Inventors: Michael Lelah, Arlington Heights, IL (US); Paul Mulhauser, New York, NY (US); Steven A. Rye, Cape Coral, FL (US)

(73) Assignee: Mercola.com Health Resources, LLC, Hoffman Estates, IL (US)

(21) Appl. No.: 15/376,870

(22) Filed: Dec. 13, 2016

Related U.S. Application Data
(60) Provisional application No. 62/270,736, filed on Dec. 22, 2015.

(51) Int. Cl. C02F 1/461; A61K 9/00; C01B 13/02; B67D 1/10; C25B 9/06; B67D 3/00; B67D 1/00; A61K 33/00; C25B 1/04

(52) U.S. Cl. CPC C02F 1/4618; A61K 33/00; A61K 9/0095; C01B 13/0207; C25B 1/04; C25B 9/06; B67D 3/0038; B67D 1/0014; B67D 1/10; C02F 2307/10; C02F 2209/06; C02F 2201/4618; B67D 2210/0001

(57) ABSTRACT

A hydrogen generation device and method are provided. The device includes componentry for separating an input flow of water such as tap water into separate hydrogen enriched and oxygen enriched flows. These flows are then recombined to produce a hydrogen enriched flow with a substantially neutral pH.

### CROSS-REFERENCE TO RELATED PATENT APPLICATIONS

This patent application claims the benefit of U.S. Provisional Patent Application No. 62/270,736, filed Dec. 22, 2015, the entire teachings and disclosure of which are incorporated herein by reference thereto.

### FIELD OF THE INVENTION

This invention generally relates to water treatment devices, and more particularly to water ionizers.

### BACKGROUND OF THE INVENTION

Water ionizers are devices which use an electrolysis process to generate molecular hydrogen gas (H2) and molecular oxygen gas (O2). The electrolysis process splits the water molecules to form separate hydrogen and oxygen ions which respectively combine to form molecular hydrogen gas (hydrogen) and molecular oxygen gas (oxygen). Consumption of hydrogen gas has been associated with a variety of health benefits. It has become recognized that the therapeutic benefits of hydrogen therapy using hydrogen enrichted water are not derived from an increased pH, but rather from the molecular hydrogen enrichment of the water.

Commercially available water ionizers produce approximately 0.5 ppm hydrogen gas. This amount has been observed to yield sufficient hydrogen to derive health benefits, which have been observed at as low as 0.08 ppm. Saturation of hydrogen gas in water is 1.6 ppm.

Such water ionizers are typically connected to a water supply, for example with a diverter adaptor affixed onto a faucet. A single conduit of water is directed into an electrolysis chamber, through which the single stream of tap water is split by an applied electric current into two separate streams, one alkaline and the other acidic. The alkaline stream contains hydrogen. The desired alkaline water is directed to an outlet, such as a spigot, for consumption. The undesired acidic water containing oxygen is directed through a waste conduit, typically freely draining into an adjacent sink. These devices also can reverse the process, producing oxygen enriched acid water as the desired stream and hydrogen enriched alkaline water as the waste stream. As used herein, “hydrogen enriched” water means water containing a concentration of H2 as a result of electrolysis. Similarly, “oxygen enriched” water as used herein means water containing a concentration of O2 as a result of electrolysis.

Water ionizers are alternatively also connected directly to water supply plumbing as a water source and the waste output may be plumbed directly into a waste drain.

In other devices, a batch type process may be used. Water is poured into a pot-like container, which internally has two electrodes separated by a filter membrane. On application of an electric current, the same separation process occurs across the membrane. At the end of the batch process, two separate solutions are created. At the cathode, hydrogen enriched alkaline water is created. At the anode, oxygen enriched acid water is created. The two are separated by the membrane and can be poured out of the container separately.

The ionizers also typically include water filters through which the incoming water is filtered of impurities, prior to electrolysis.

In a typical conventional water ionizer, tap water enters the electrolysis chamber through a single flow conduit. The tap water typically has a neutral pH of 7. The electrolysis unit, within a conventional water ionizer, has a chamber containing electrodes. Electrical current enters the water filled chamber through negatively charged electrodes, known as cathodes. The current flows through the contained water and exits through positively charged electrodes, known as anodes.

Positive ions (cations) gather at the negative electrodes (cathodes), increasing the pH to create alkaline water and generate hydrogen ions (H+) which immediately combine to form hydrogen gas (hydrogen) which is typically dissolved in the water (hydrogen enriched alkaline water). Negative ions (anions) gather at the positive electrodes (anodes), lowering the pH to create acid water and generate hydroxyl ions (OH−)which immediately react with water to form oxygen gas (oxygen) which is typically dissolved in the water (oxygen enriched acid water).

The electrolysis chamber includes a membrane/filter between the anodes and cathodes to separate the alkaline and acid portions of contained water, which are then diverted to exit the chamber through two separate flow conduits.

While the above-described water ionizers have proven beneficial in use, they are inherently inefficient due to the draining away of the oxygen enriched acidic water. Further, the hydrogen enriched water delivered is typically higher in pH. Accordingly, there is a need in the art for a water ionizer which, on the one hand, efficiently uses the water it draws from its source, and on the other hand delivers a more pH neutral water for consumption, despite being enriched with hydrogen as described above.

The invention provides such a water ionizer. These and other advantages of the invention, as well as additional inventive features, will be apparent from the description of the invention provided herein.

### BRIEF SUMMARY OF THE INVENTION

Embodiments of the invention herein provide a method and a device that boosts the hydrogen content of tap water while maintaining substantially neutral pH. Such a device may be preferably useful in a home or commercial kitchen, however may also be used in other residential or commercial applications or in any other such place where tap water is drawn for consumption or other uses. hydrogen enriched water has also been recognized, for example, to have benefits for cleaning soiled surfaces. Indeed, there are many beneficial uses for hydrogen enriched water for which embodiments of this invention may be useful.

The device may preferably be configured, for example, for countertop or table top use but may additionally be configured for other free standing or plumbed installations or other uses as may be envisioned by one of ordinary skill in the art.

The device may be configured to treat small volumes of water, for example for home usage. The device is also scalable, for example for high volume commercial applications. The hydrogen enriched water may be used for medical or non-medical applications.

As discussed above, in a conventional water ionizer device, generally neutral pH tap water passes through an electrolysis chamber which separates the water to be expelled from two outlet channels. (Channel is used to describe, for example, a conduit, tube, or other controlled water flow path) One outlet channel expels hydrogen enriched water for consumption and the other outlet expels oxygen enriched water, typically to a waste drain.

As discussed above, conventional water ionizers dispense only the hydrogen enriched high pH portion of the processed water through one conduit and discard the separated oxygen enriched low pH portion of the processed water through a second conduit. Unlike such conventional devices, embodiments of the invention herein provide a device which instead recombines the hydrogen enriched portion with the oxygen enriched portion to dispense hydrogen enriched water with approximately neutral pH for consumption.

As discussed above, conventional water ionizing products and wisdom advocate the use of hydrogen enriched water for medical and other applications. One unique aspect of embodiments of this invention is the generation of hydrogen enriched substantially neutral pH water, where the neutrality (or approximate neutrality) of the water is achieved by combining the acid and alkali streams, resulting in hydrogen enriched substantially neutral pH water. hydrogen enriched alkali water has been previously used for medical or therapeutic purposes, where the health benefits have been variously attributed to the alkalinity of the water and/or the presence of hydrogen dissolved gasses. One advantage of embodiments of the present invention is thus the creation of hydrogen enriched water which is substantially neutral in pH, so the medical/health benefits are directly attributable to the dissolved hydrogen gasses are gained and there are no taste or other defects associated with an alkaline pH.

In one aspect, embodiments of the invention provide a device for modifying neutral pH tap water into hydrogen enriched water with substantially neutral pH. An embodiment according to this aspect includes an inlet. An electrolytic cell is in fluid communication with the inlet. The electrolytic cell includes a chamber containing one or more each of both an anode and a cathode with a membrane between each, for the purpose of splitting the neutral pH water into separate hydrogen enriched water with a higher pH and oxygen enriched water with a lower pH. The device also includes a flow combiner connected to the chamber and configured to bring together the flow of hydrogen enriched water with the oxygen enriched water to form a combined hydrogen enriched water mixture with a substantially neutral pH. The device also includes an outlet connected to the flow combiner through which the hydrogen enriched water mixture is dispensed for consumption.

The device may be configured as a countertop device and is not connected to a pressurized water source such as a faucet. The water flow along a flow path through the device may be gravity driven, or may be driven by a pump.

The device may also include a replaceable water filter module is insertable into the flow path between the inlet and the electrolytic cell. The replaceable water filter module is situated upstream from the pump relative to a flow path through the device.

The inlet, electrolytic cell, and flow combiner are housed within a housing, with the outlet extending out of the housing. A space may be provided adjacent the housing below the outlet for receipt of a container such that the container can receive the hydrogen enriched water mixture.

The device may also include a flexible inlet conduit which is connectable at a distal end to receive tap water from a water source such as a faucet, and which is connectable at a proximal end to the inlet. With such a configuration, a manually activated means to initiate and to control the amount of water to flow through the electrolytic cell to be dispensed may also be employed.

In another aspect, embodiments of the invention provide a device for modifying neutral pH tap water into hydrogen enriched water with substantially neutral pH, and for dispensing the modified water. An embodiment according to this aspect includes an inlet configured to receive water via a conduit which is attached to a water source such as a faucet. The device also includes an electrolytic cell, in fluid communication with the inlet, with a chamber containing one or more each of both an anode and a cathode with a membrane between each, for the purpose of splitting the neutral pH water into separate hydrogen enriched water with a higher pH and oxygen enriched water with a lower pH. A flow combiner is connected to the chamber and configured to bring together the flow of hydrogen enriched water with the oxygen enriched water to form a combined hydrogen enriched water mixture with a substantially neutral pH. The device also includes an outlet through which the hydrogen enriched water mixture is dispensed for consumption.

In an embodiment according to this aspect, the water flow along a flow path through the device is driven by a pump. A replaceable water filter module is insertable into the flow path between the inlet and the electrolytic cell. The replaceable water filter module may be situated upstream from the pump relative to a flow path through the device.

The inlet, electrolytic cell, and flow combiner are housed within a housing, and wherein the outlet extends out of said housing. A space may be provided adjacent the housing below the outlet for receipt of a container such that the container can receive the hydrogen enriched water mixture.

In another aspect, embodiments of the invention provide a method for generating hydrogen rich water having a substantially neutral pH using a device. An embodiment of such a method includes introducing water from a source into an inlet of the device. The method also includes passing the water from the source through an electrolytic cell such that the water is separated into hydrogen enriched water, and oxygen enriched water. The method also includes recombining the separated hydrogen enriched water and oxygen enriched water to form a hydrogen enriched water mixture having a neutral pH.

The method also includes dispensing the hydrogen enriched water mixture from an outlet of the device. The step of dispensing includes dispensing the hydrogen enriched water mixture from the outlet situated in a space adjacent the device, the space configured to receive a container for receipt of the hydrogen enriched water mixture.

In one embodiment, the step of passing the water from the source through the electrolytic cell is done via gravity. In another embodiment, the step of passing the water from the source through the electrolytic cell is done via a force provided by a pump.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1A is a schematic illustration showing an exemplary conventional water electrolysis chamber; the chamber containing one or more each of anodes & cathodes and having two output channels—one output channel for oxygen enriched water and a second outlet channel for hydrogen enriched water.

FIG. 1B is another schematic illustration showing an exemplary conventional water electrolysis chamber, with fluid connection through water filters upstream of the electrolysis chamber, again having two output channels—one outlet channel for oxygen enriched water flow and a second outlet channel for hydrogen enriched water flow.

FIG. 1C is another schematic illustration showing another exemplary conventional water electrolysis chamber, with fluid connection through water filters, again having two output channels—one output channel for oxygen enriched water flow (often, as shown here, directed to waste) and a second outlet channel for hydrogen enriched water flow (often, as shown here, directed for use). The inlet flow channel is alternatively shown here connected, for example, onto a faucet or spigot, and the hydrogen enriched output flow channel is alternatively shown here positioned to replace the traditional tap water flow.

FIG. 2A is a schematic illustration showing an electrolysis chamber containing multiple alternately spaced apart positively and negatively charged electrodes. Each electrode is contained within a separate cell, separated by semi-permeable ion-exchange filter membranes. Tap water, with generally neutral pH, is directed through flow channels into each of the electrode containing cells within the electrolysis chamber. Acidic water, created in the one or more cells containing positively charged electrode cells, is expelled through a first output channel. Hydrogen enriched water, created in the one or more negatively charged electrodes, is directed through a second output channel.

FIG. 2B is a schematic illustration of an exemplary embodiment of the instant invention, with an electrolysis chamber as shown in FIG. 2A, instead showing the oxygen enriched and hydrogen enriched output flow channels inter-connected in a manifold-like configuration via a flow combiner so as to produce a combined substantially neutral pH mixture which is then dispensed through a single output flow channel. As can be seen from inspection of FIG. 2B, the flow combiner is thus the interconnected flow channels immediately upstream from the outlet channel.

FIG. 3 is a schematic illustration of an exemplary embodiment of the instant invention, showing an electrolytic ionizer device with combined output flow channels as shown in FIG. 2B. The electrolytic ionizer device is shown here in fluid connection with a funneled water inlet aperture above, configured to gravity feed input water into and through the electrolytic ionizer device. The separate outlet channels (from the electrolytic ionizer device) are shown connected into a single output flow channel via a flow combiner in a similar manner as that shown in FIG. 2B, so as to create a combined mixture of substantially neutral pH water. The single outlet flow channel is shown positioned above a void space, provided to accept a water receiving receptacle, for example, a drinking glass or a water container.

FIG. 4 is a schematic illustration of an exemplary embodiment of the invention which is a variation of schematic FIG. 3, comprising an electrolytic ionizer device with combined output channels to a single outlet and a water input aperture, additionally including a water filtration module in fluid communication between the input channel and the electrolysis chamber. An outline profile shows an exemplary enclosure configuration for illustrative purposes only.

FIG. 5 is a schematic illustration of an exemplary embodiment of the invention which is a variation of schematic FIG. 4, comprising an electrolytic ionizer device with combined output channels, in fluid communication with a water input aperture and a water filtration module, with addition of a pump to drive water through the system.

FIG. 6 is a schematic illustration of an exemplary embodiment of the invention which is a variation of schematic FIG. 5, comprising an electrolytic ionizer device with combined output channels, in fluid communication through a water filtration module and a pump, with an input conduit connectable to a faucet or other source of tap water.

FIG. 7 is a schematic illustration of an exemplary embodiment of the invention which is a variation of schematic FIGS. 3, 4, 5, 6 in which the hydrogen enriched water and oxygen enriched water exiting the electrolytic ionizer device are directed into separate fluid holding containers, from which they are subsequently controllably dispensed together, through a flow control valve, as a combined substantially neutral pH water mixture, maintaining the benefit of hydrogen enrichment. Such a configuration may additionally include a pre-filter module and/or a pump.

FIG. 8 is an illustration of an exemplary embodiment of the invention which is a schematic illustration showing an electrolytic ionizer device having two output conduits, with one output conduit expelling oxygen enriched water and a second outlet conduit expelling hydrogen enriched water. Both outlet conduits are in close proximity above a space provided to position a water receiving receptacle, for example a drinking glass, enabling the two expelled water volumes to combine and mix together into the receiving receptacle, as hydrogen enriched water with a substantially neutral pH level. The flow combiner in this embodiment is thus container itself

FIG. 9 is an illustration of an exemplary embodiment of the invention which is a schematic illustration showing an electrolytic ionizer device having two output conduits, with one output conduit expelling oxygen enriched water and a second outlet conduit expelling hydrogen enriched water. Both outlet conduits flow into a common chamber serving as a flow combiner for recombining the flows and positioned above a space provided to position a water receiving receptacle, for example a drinking glass. This recombined water may then be dispensed through an output channel into the drinking glass.

FIG. 10 is an illustration showing an abstract exterior form as may be embodied to shroud an interconnected system through which water is passed to become hydrogen enriched, as shown for example in FIG. 3, 4, or 5.

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

Turning now to the drawings, one advantage of embodiments described herein is the production and delivery of hydrogen enriched water with substantially neutral pH for consumption. As used herein, the phrase “substantially neutral pH” means pH values ranging from 6.5 to 7.5. One embodiment of the invention is intended for home or office use, for example used as a counter top device. However, the device and device process may also be used for other commercial applications.

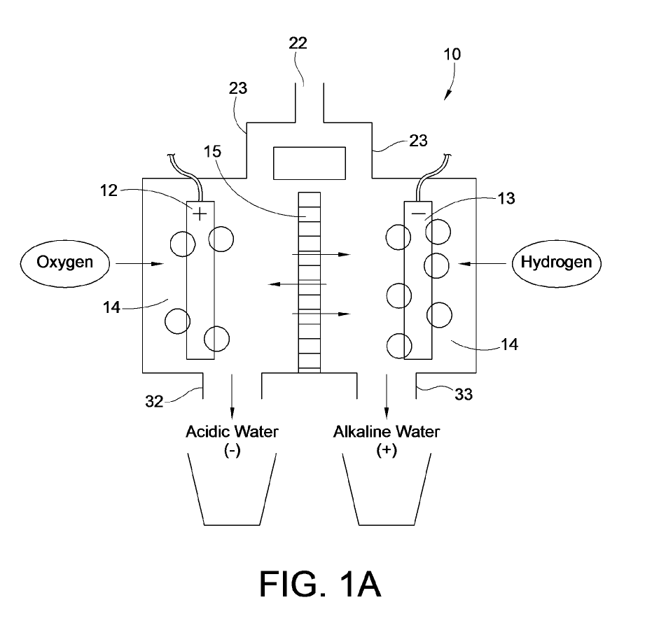

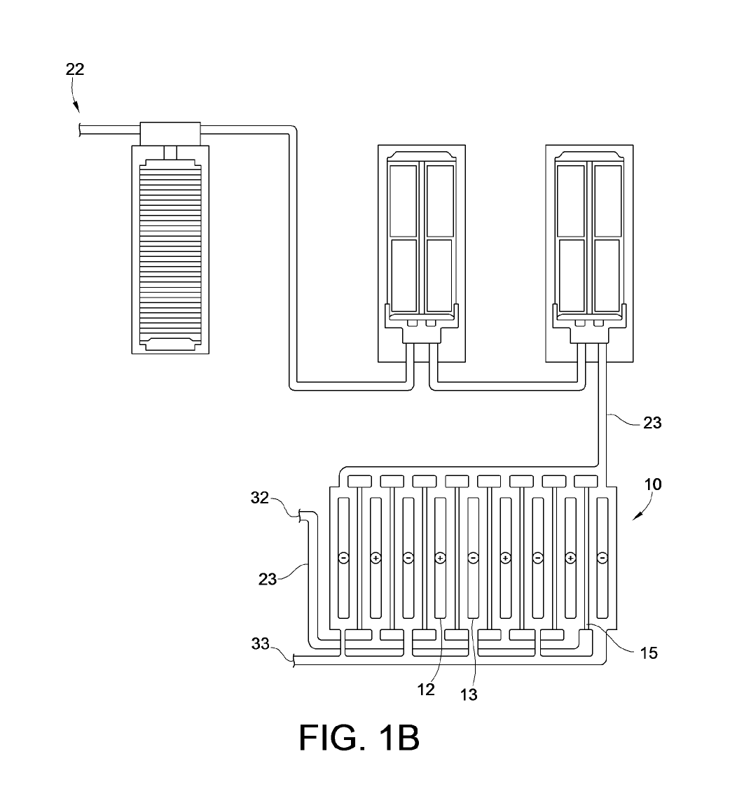

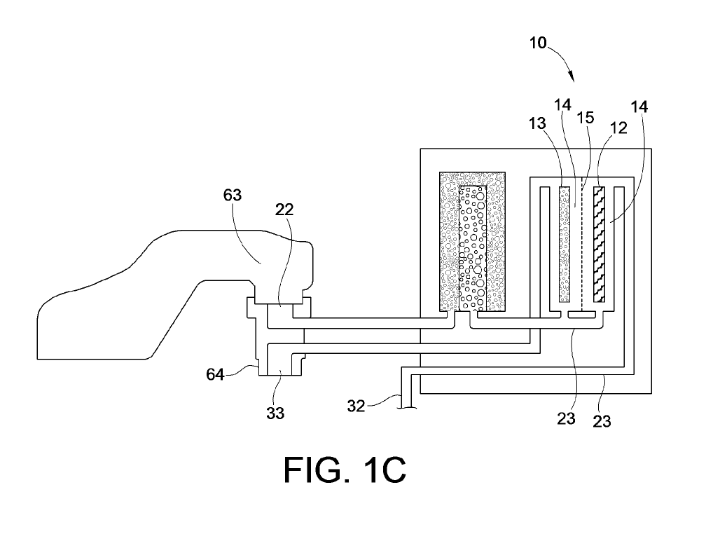

FIG. 1A, 1B, 1C and 2A schematically illustrate examples of the basic configuration of conventional water ionizer devices as used to derive hydrogen enriched water from tap water. Tap water is introduced through a water inlet flow channel 22 to be processed within an electrolytic ionizer device 10. The introduced water is directed through flow channels 23, into separate electrode containing cells, within an electrolysis chamber 11. Each of the electrode containing cells alternately contains either a positively charged electrode (anode) 12 or a negatively charged electrode (cathode) 13.

Electrodes used in conventional electrolytic ionizer devices 10 are often configured as plates and are most often made with or plated with inert metals (for example platinum or titanium) that are not dissolved by electrolytic reactions. Ionic flow between the anodes 12 and cathodes 13 changes the pH of the surrounding water. The water within the anode 12 containing cells 14 becomes more acidic (lower pH and also becomes enriched with oxygen gas molecules). The undesired acidic water is typically directed through flow channels 23 and to (a waste) outlet channel 32. The water within the cathode 13 containing cells 14 becomes more alkaline (higher pH and also enriched with hydrogen gas molecules). The Hydrogen enriched alkaline water is directed through flow channels 23 to a (dispensing) outlet channel 33.

As shown in FIGS. 1A and 1B, the water inlet flow channel 22, may be connected remotely, for example, onto a sink faucet or otherwise connected into a tap water plumbing source. The water inlet flow channel in FIG. 1C is schematically shown connected to a faucet/spigot 63, via a faucet/spigot type adaptor fitting 64.

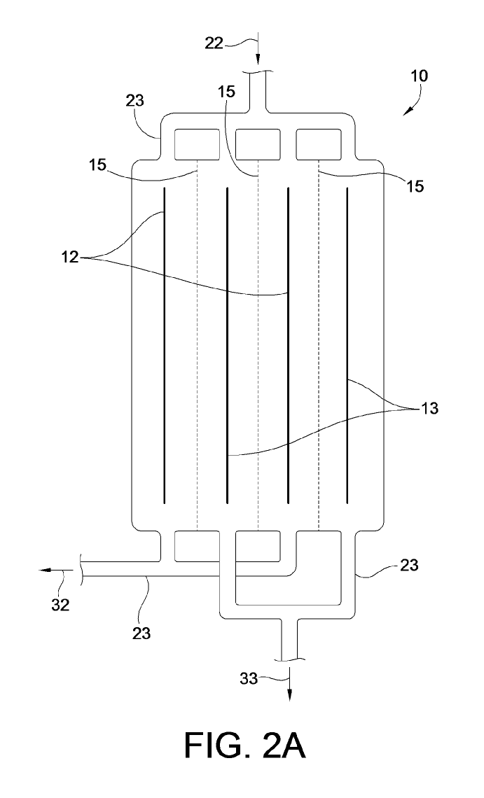

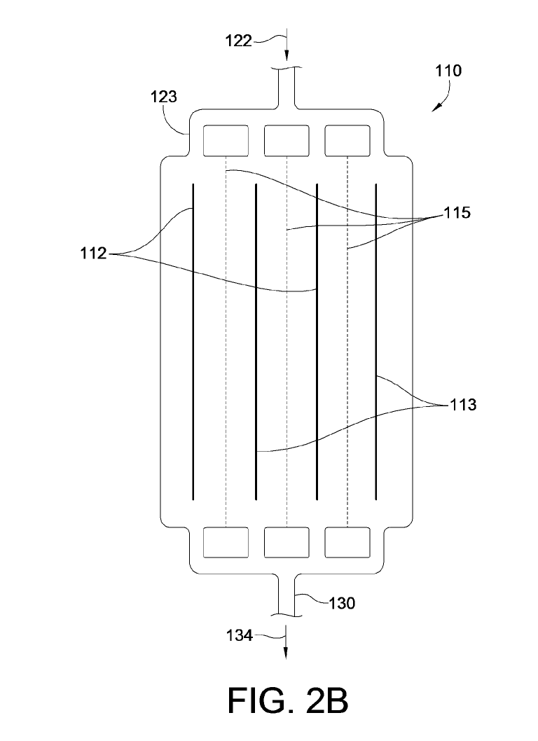

FIG. 2B schematically illustrates an embodiment of a unique and novel improvement over how conventional water ionizers function, as exemplified comparatively above by FIG. 2A, according to the teachings of the present invention. The improved embodiment shown in FIG. 2B uses an electrolytic ionizer device 110 to separate water passing through it into an oxygen enriched water within those cells containing anodes 112, and into a hydrogen enriched water within those cells containing cathodes 113. Water enters electrolytic ionizer device through inlet channel 122, and may be separated into separate flows via flow channels 123. These separate flows are then exposed to chambers containing either a cathode or an anode separated by membranes as described above.

However, as further shown in Fig, 2B, rather than expelling the oxygen enriched water separately from the hydrogen enriched water, both are instead combined and mixed together through a connected a flow combiner 130, to then be dispensed as a combined hydrogen enriched substantially neutral pH water mixture through a combined outlet 134 for dispensing. As illustrated, flow combiner 130 is a manifold construction, but may also be a chamber into which the output flows from electrolytic ionizer device 110 flow into and are combined. Embodiments of the invention thus pertain to the unique and novel (and counter-intuitive) combining of the output flows to achieve a hydrogen enriched water mixture with substantially neutral pH.

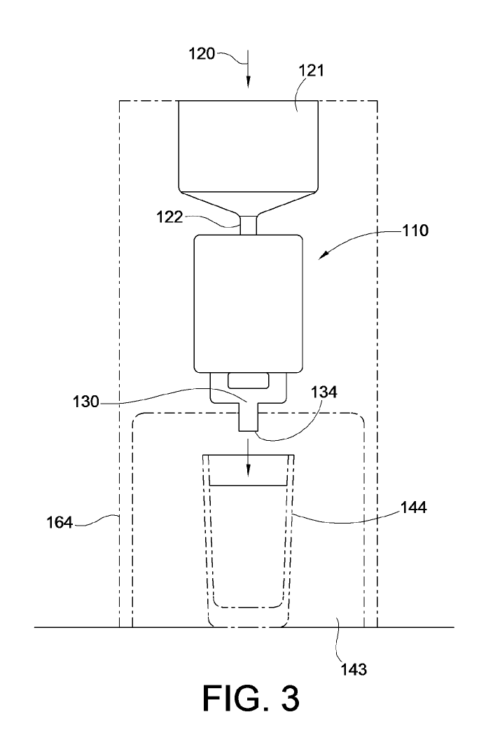

In an embodiment of the invention, as further schematically shown in FIG. 3, a controllable volume of tap water, such as a glass of water, may be introduced through a water inlet 120, as shown configured, for example, leading to an open or funneled cavity 121. The introduced tap water may gravity feed through inlet flow channel 122, into and through electrolytic ionizer device 110, through which the tap water is separated into hydrogen enriched water and oxygen enriched water.

The separated water then flows from the electrolytic ionizer device 110, through flow combiner 130, through which the flows combine to form a substantially neutral pH water mixture with maintained hydrogen enrichment. The combined mixture then flows through a combined outlet flow orifice 134, positioned above a void space 143, configured to accept a container 144 (such as a drinking glass or drinking water container) into which the hydrogen enriched water is dispensed. The device shown in FIG. 3 is configured as a ‘pour-through system’ to process a freshly introduced controllable volume of tap water with each individual use. The spacing of anodes 112, cathodes 113 and membranes 115 may be configured for optimal gravity flow through (see e.g. FIG. 2B).

In such an embodiment as shown in FIG. 3, a user will pour a desired volume of water, for example a filled drinking glass, into an inlet 120 leading to funneled cavity 121 at the top of the device. The same or another drinking glass will be placed into a void space 143 provided below the outlet flow channel 134. Upon manual or automatic actuation, the water will flow or be pumped through the electrolytic ionizer device 110. The hydrogen enriched and oxygen enriched outputs will be recombined through a connected output channel 130 forming a hydrogen enriched substantially neutral pH mixture, to be dispensed through outlet 134. In this manner, a given volume of water, introduced into the device, will be processed to produce and dispense hydrogen enriched water with substantially neutral pH for consumption.

As may also be seen in FIG. 3, electrolytic ionizer device 110, funneled cavity 121, flow combiner 130 and their associated conduits are housed within a housing 164. Outlet 134 is exposed in a void space 143 adjacent housing. The placement of outlet 134 is such that a container may be positioned within void space 143 to collect the output flow from outlet 134.

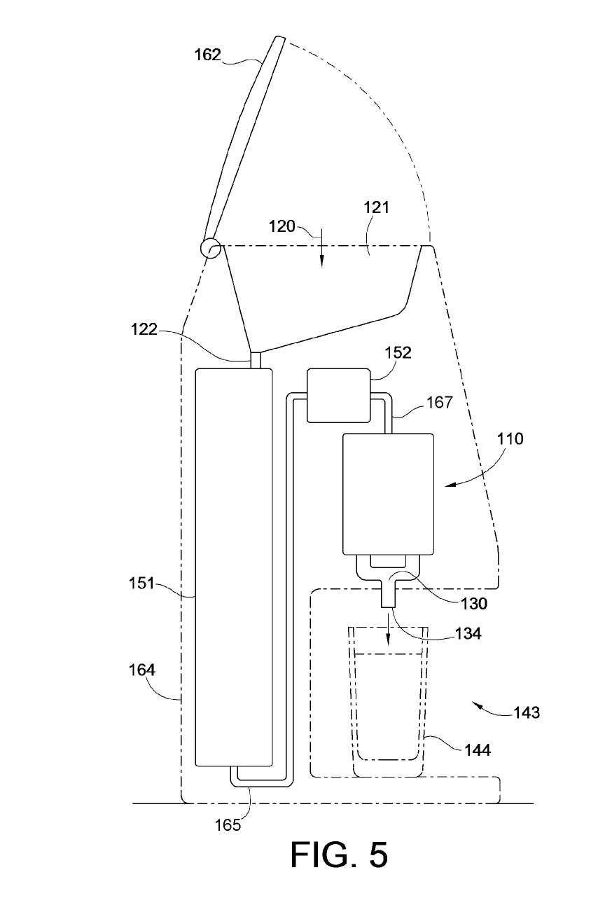

Additionally, as schematically shown in FIG. 3, a funneled cavity 121 may be positioned at or near the top of the device. The cavity 121 may have an openable protective lid 162, for example hinged, to cover inlet 120 when not in use (as shown ahead in FIG. 5). Inlet 120 and cavity 121 are configured to receive tap water, for example poured in from a container, such as a drinking glass, pitcher, or other vessel. Covers other than hinged covers are of course contemplated, such as slidable, rotatable, or removable covers as well.

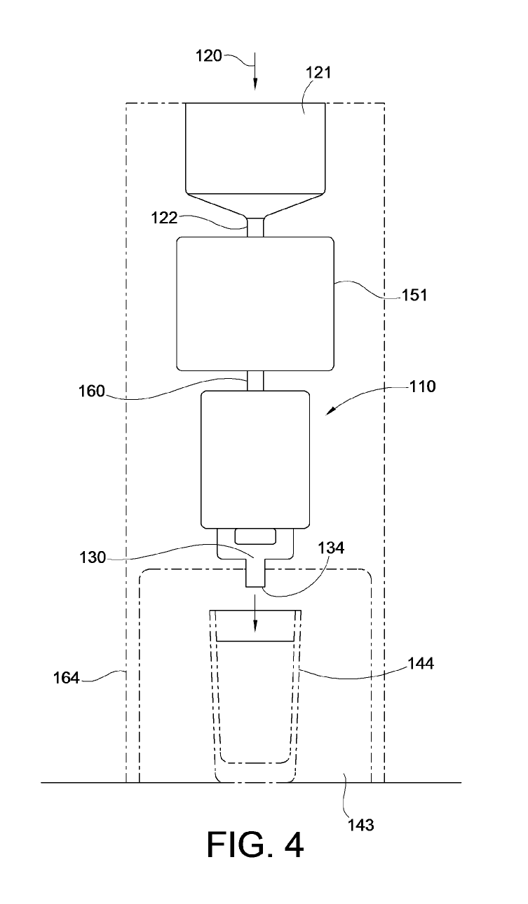

FIG. 4 shows the basic operative system as schematically illustrated in FIG. 3, including inlet 120 leading to cavity 121, electrolytic ionizer device 110 in fluid communication with inlet 120 via flow channel 122, flow combiner 130 connected on the outlet side of electrolytic ionizer device 110, and an outlet 134, with the additional inclusion of a replaceable water filtering module or modules 151 in fluid communication between the inlet 120 and the electrolytic ionizer device 110 to purify the inlet water of contaminants. Filters may include, for example: replaceable or non-replaceable fiber, activated charcoal, calcium, anti-microbial, chlorine reduction, or other filter types, or combination thereof. Those of skill in the art will recognize, however, that the invention is not limited to any particular type of filter. Any filter suitable for achieving the filtration function of water into the system may be utilized or no filter at all, as introduced above.

FIG. 5 schematically shows the basic operative system as illustrated in FIG. 4, with inclusion of a water pump 152. A flow channel 165 connects filter module or modules 151 with water pump 152, and a flow channel 167 connects water pump 152 with electrolytic ionizer device 110. The water pump 152 enables input water to be controllably driven through the device at optimal pressures, rates, and volumes to achieve maximal desired water throughput and hydrogen benefit. The water pump may be powered through an electrical source such as a home's electrical system, or alternatively, may be battery powered. The inclusion of a pump 152 to drive fluid flow through the device facilitates ability to alternatively configure and proportion the overall fluidic system.

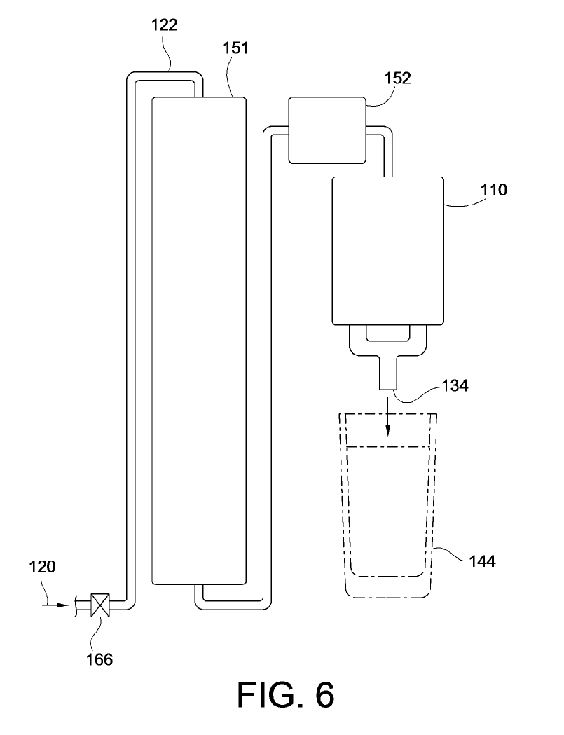

FIG. 6 schematically illustrates a variation of the embodiment shown in FIG. 5 in which the water inlet flow channel 122 is intended to be alternatively connected (not shown) into to a remote tap water source, e.g. tap water plumbing. The water inlet flow channel 122, may alternatively be connected through a faucet/spigot adaptor 166 to divert tap water from the faucet/spigot similar to what is illustrated for example in FIG. 1C. In this configuration, water inlet flow channel 122 may be configured as a flexible hose or the like for ease of connection.

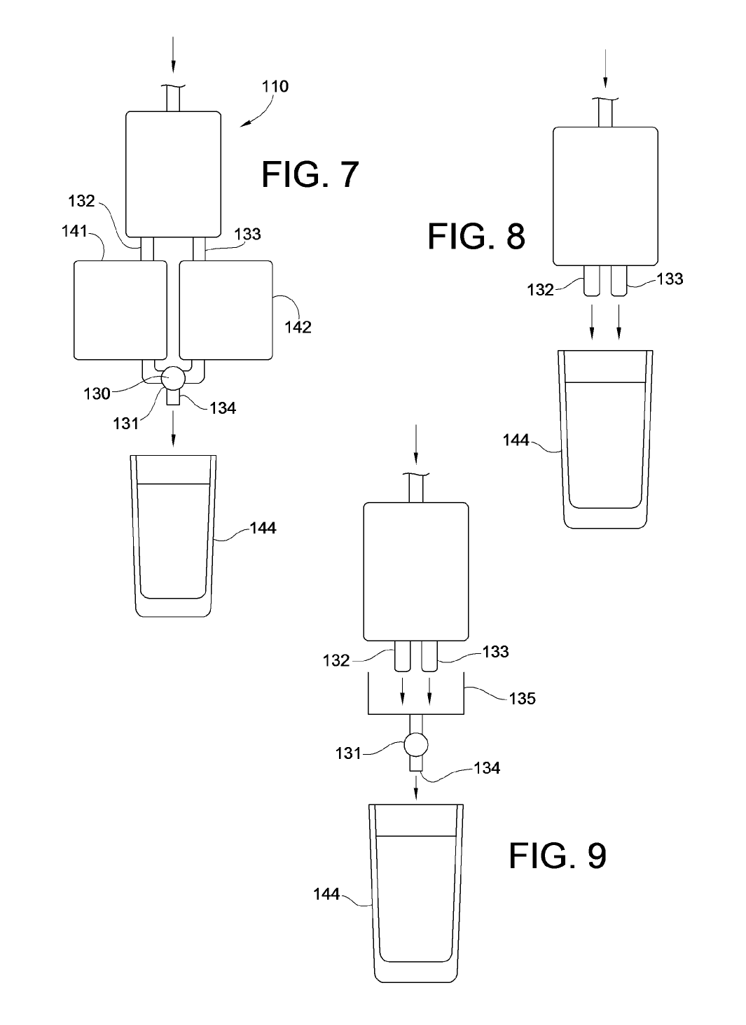

FIG. 7 shows an alternative embodiment of the invention in which the oxygen enriched water, flowing through one or more outlet channels 132 from the electrolytic ionizer device 110, may flow into an oxygen enriched water containing chamber 141. The hydrogen enriched water, flowing through outlet flow channels 133 from the electrolysis chamber 110, may flow into a hydrogen enriched water containing chamber 142. By opening a water flow control valve 131 in flow combiner 130, the separated water components may subsequently be withdrawn from the holding chambers 141 and 142, and dispensed through outlet 134 as an hydrogen enriched mixture of substantially neutral pH water.

FIG. 8 shows an alternative embodiment in which one outlet flow channel 132, with oxygen enriched water flow, is relatively adjacent to or adjoining with a second outlet flow channel 133, with hydrogen enriched water flow. Both outlet channels 132 and 133 are positioned above container 144, such that the two flows combine and form a substantially neutral pH mixture in container 144. In this manner the user may control the amounts of resulting hydrogen enriched substantially neutral pH water mixture to be dispensed as desired. This alternative embodiment may be more efficient if connected to a tap water supply than if configured as a ‘pour through system’.

In yet another alternative embodiment as shown in FIG. 9, the outlet flow channels 132, 133 may both flow into a common water holding chamber 135 to form a substantially neutral pH hydrogen enriched water mixture. By opening a water flow control valve 131, the hydrogen enriched mixture may be dispensed through a single outlet flow channel 134 as desired.

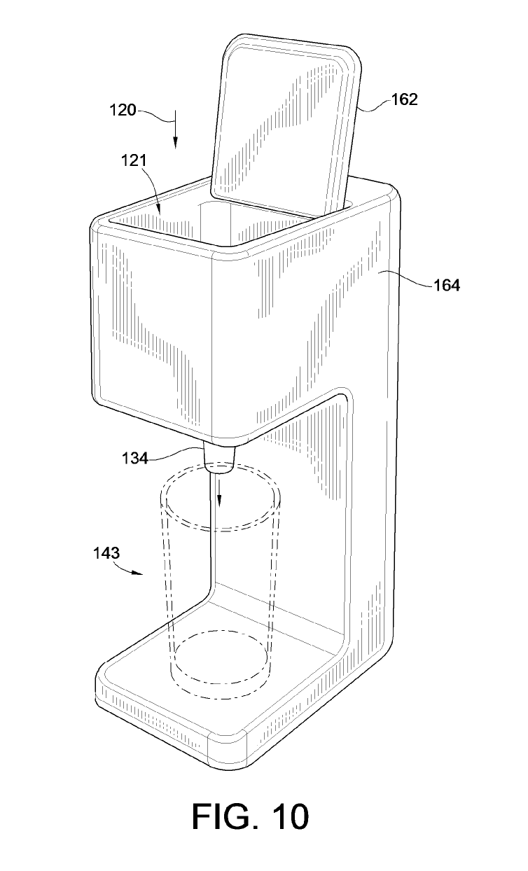

FIG. 10 illustrates an abstract exterior form of housing 164 as may be embodied to shroud an interconnected system through which water is passed to become hydrogen enriched, as shown for example in FIG. 3, 4, or 5.

Not shown in FIG. 10, but as shown generally described though the above Figures, the water flow through this exemplary embodiment, or variations of it, may be gravity or pump 152 driven. The embodiment may also include a filter or filters 151. The embodiment, or variations of it, may alternatively substitute a water inlet flow channel 122 from a remote source of tap water, in lieu of the open water inlet aperture 121 and cover 162.

Referring generally to the figures described, the operative system may also include electronic controls and control displays (not shown) to facilitate user adjustable optimization of system variables, such as electrolytic device power and water flow pressure, rate and output volume. Such ability to adjust system variables may be beneficial to accommodate local variations in available water quality. An automatic water sensor or float switch or other manually activated switch may be included to start the electrolytic cell function upon introduction of water. A sensor may also be included to terminate power to the electrolytic cell upon process completion. A flow valve may be included to controllably open or close dispensing flow through the outlet. A flow valve may also be used to controllably restrict or temporarily close flow through the electrolytic chamber, as useful to optimize dwell time for the water to pass between the electrodes for ionization. Sensors may also be included for the purpose of closing the outlet flow and to provide a visual or audible indication, should a glass not be present under the outlet during system activation.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A device for modifying neutral pH tap water into hydrogen enriched water with substantially neutral pH, comprising:
an inlet;
an electrolytic cell, in flowable communication with the inlet, with a chamber containing one or more each of both an anode and a cathode with a membrane between each, for the purpose of splitting the neutral pH water into separate hydrogen enriched water with a higher pH and oxygen enriched water with a lower pH;
a flow combiner connected to the chamber and configured to bring together the flow of hydrogen enriched water with the oxygen enriched water to form a combined hydrogen enriched water mixture with a substantially neutral pH; and
an outlet connected to the flow combiner through which the hydrogen enriched water mixture is dispensed for consumption.

2. The device of claim 1, wherein the device is configured as a countertop device and is not connected to a pressurized water source such as a faucet.

3. The device of claim 1, wherein the water flow along a flow path through the device is gravity driven.

4. The device of claim 1, wherein the water flow through the device along a flow path is driven by a pump.

5. The device of claim 1, wherein a replaceable water filter module is insertable into the flow path between the inlet and the electrolytic cell.

6. The device of claim 5, wherein the replaceable water filter module is situated upstream from the pump relative to a flow path through the device.

7. The device of claim 1, wherein the inlet, electrolytic cell, and flow combiner are housed within a housing, and wherein the outlet extends out of said housing.

8. The device of claim 7, wherein a space is adjacent the housing below the outlet for receipt of a container such that the container can receive the hydrogen enriched water mixture.

9. The device of claim 1, further comprising a flexible inlet conduit which is connectable at a distal end to receive tap water from a water source such as a faucet, and which is connectable at a proximal end to the inlet.

10. A device for modifying neutral pH tap water into hydrogen enriched water with substantially neutral pH, comprising:
an inlet configured to receive water via a conduit which is attached to a water source such as a faucet;
an electrolytic cell, in fluid communication with the inlet, with a chamber containing one or more each of both an anode and a cathode with a membrane between each, for the purpose of splitting the neutral pH water into separate hydrogen enriched water with a higher pH and oxygen enriched water with a lower pH;
a flow combiner connected to the chamber and configured to bring together the flow of hydrogen enriched water with the flow of oxygen enriched water to form a combined hydrogen enriched water mixture with a substantially neutral pH; and
an outlet in fluid communication with the flow combiner through which the hydrogen enriched water mixture is dispensed for consumption.

11. The device of claim 10, wherein the water flow along a flow path through the device is driven by a pump.

12. The device of claim 11, wherein a replaceable water filter module is insertable into the flow path between the inlet and the electrolytic cell.

13. The device of claim 12, wherein the replaceable water filter module is situated upstream from the pump relative to a flow path through the device.

14. The device of claim 10, wherein the inlet, electrolytic cell, and flow combiner are housed within a housing, and wherein the outlet extends out of said housing.

15. The device of claim 14, wherein a space is adjacent the housing below the outlet for receipt of a container such that the container can receive the hydrogen enriched water mixture.

16. A method for generating hydrogen rich water having a substantially neutral pH using a device, the method comprising the steps of:
introducing water from a source into an inlet of the device;
passing the water from the source through an electrolytic cell such that the water is separated into hydrogen enriched water, and oxygen enriched water;
recombining the separated hydrogen enriched water and oxygen enriched water to form a hydrogen enriched water mixture having a neutral pH.

17. The method of claim 16, further comprising dispensing the hydrogen enriched water mixture from an outlet of the device.

18. The method of claim 17, wherein the step of dispensing includes dispensing the hydrogen enriched water mixture from the outlet situated in a space adjacent the device, the space configured to receive a container for receipt of the hydrogen enriched water mixture.

19. The method of claim 16, wherein the step of passing the water from the source through the electrolytic cell is done via gravity.

20. The method of claim 16, wherein the step of passing the water from the source through the electrolytic cell is done via a force provided by a pump.
